(19) United States
(12) Patent Application Publication
(10) Pub. No.: US 2003/0083503 A1
(43) Pub. Date: May 1, 2003

(54) Synthesis of acylated keto amides from sulfonyl amides

(76) Inventors: Doug E. Frantz, Downingtown, PA (US); Lisa F. Frey, Somerset, NJ (US); Karen Marcantonio, New York, NY (US); Jerry Anthony Murry, New York, NY (US); Richard Tillyer, Cranford, NJ (US)

Correspondence Address: MERCK AND CO INC, P O BOX 2000, RAHWAY, NJ, 070650907

(21) Appl. No.: 10/177,870

(22) Filed: Jun. 20, 2002

Related U.S. Application Data
(60) Provisional application No. 60/299,653, filed on Jun. 20, 2001.

(51) Int. Cl. C07C 269/00; C07C 273/00; C07C 231/06; C07D 213/54

(52) U.S. Cl. 546/314; 560/24; 564/124; 564/47

(57) ABSTRACT

Acylated ketoamides are prepared from sulfonyl amides and aldehydes or their related derivatives by a step of

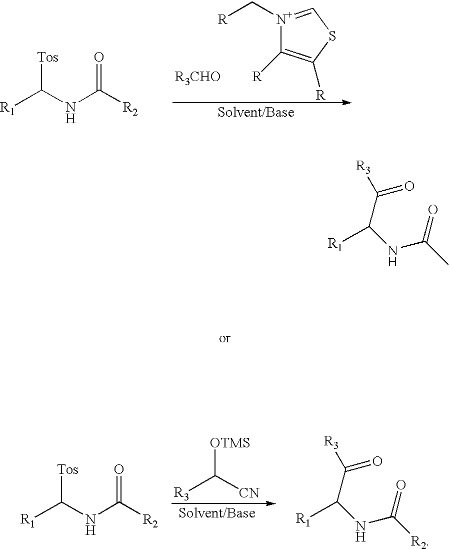

### BACKGROUND OF THE INVENTION

1. Field of the Invention

The present invention is directed to a method to make acylated keto amides. In particular, this invention is directed to a method to make acylated keto amides from sulfonyl amides.

2. Related Background

The present invention relates to make acylated keto amides useful as intermediate compounds to make, for example, substituted imidazole compounds which have anti-cancer activity. Such substituted imidazole compounds are described in, for example, U.S. Pat. No. 5,717,100.

The substituted imidazole compounds demonstrate anti-cancer activity through the antagonism of the kinase, Raf. The raf genes code for a family of proteins, which can be oncogenically activated through N-terminal fusion, truncation or point mutations. RAF can be activated and undergoes rapid phosphorylation in response to PDGF, EGF, insulin, thrombin, endothelin, acidic FGF, CSF1 or TPA, as well as in response to oncoproteins v-fms, v-src, v-sis, Hras and polyoma middle T antigen. Antisense constructs which reduce cellular levels of c-Raf, and hence Raf activity, inhibit the growth of oncogene-transformed rodent fibroblasts in soft agar, while exhibiting little or no general cytotoxicity. Since inhibition of growth in soft agar is highly predictive of tumor responsiveness in whole animals, these studies suggest that the antagonism of RAF is an effective means by which to treat cancers in which RAF plays a role.

Examples of such cancers, where RAF is implicated through overexpression include cancers of the brain, genitourinary tract, lymphatic system, stomach, larynx and lung. More particularly, such examples include histiocytic lymphoma, lung adenocarcinoma and small cell lung cancers. Additional examples include cancers in which overexpression or activation of Raf-activating oncogenes (e.g., K-ras, erb-B) is observed. More particularly, such cancers include pancreatic and breast carcinoma.

Such substituted imidazoles also inhibit cytokines and the pathology, which is associated with diseases wherein cytokines are present in high levels. Cytokine mediated diseases refers to diseases or conditions in which excessive or unregulated production or activity of one or more cytokines occurs. Interleukin-1 (IL-1) and Tumor Necrosis Factor (TNF) are cytokines produced by a variety of cells that are involved in immunoregulation and other physiological conditions.

IL-1 is implicated in many disease states. Included among these diseases are rheumatoid arthritis, osteoarthritis, endotoxemia, toxic shock syndrome, acute and chronic inflammatory diseases, such as the inflammatory reaction induced by endotoxin or inflammatory bowel disease; tuberculosis, atherosclerosis, muscle degeneration, cachexia, psoriatic arthritis, Reiter's syndrome, rheumatoid arthritis, gout, traumatic arthritis, rubella arthritis and acute synovitis. Recent evidence also links IL-1 activity to diabetes.

Interleukin-1 has also been demonstrated to mediate a variety of biological activities thought to be important in immunoregulation and other physiological conditions. The known biological activities of IL-1 include the activation of T helper cells, induction of fever, stimulation of prostaglandin or collagenase production, neutrophil chemotaxis, induction of acute phase proteins and the suppression of plasma iron levels.

Excessive or unregulated tumor necrosis factor (TNF) production or activity has likewise been implicated in mediating or exacerbating rheumatoid arthritis, rheumatoid spondylitis, osteoarthritis, gouty arthritis, and other arthritic conditions, sepsis, septic shock, endotoxic shock, gram negative sepsis, toxic shock syndrome, adult respiratory distress syndrome, cerebral malaria, chronic pulmonary inflammatory disease, silicosis, pulmonary sarcosis, bone resorption diseases, reperfusion injury, graft v. host rejection, allograft rejections, fever and myalgia due to infection, cachexia secondary to infection or malignancy, cachexia secondary to acquired immune deficiency syndrome (AIDS), AIDS related complex (ARC), keloid formation, scar tissue formation, Crohn's disease, ulcerative colitis and pyresis.

Monokines, such as TNF, have also been shown to activate HIV replication in monocytes and/or macrophages. Therefore, inhibition of monokine production or activity aids in limiting HIV progression. TNF has been implicated in various roles with other viral infections, such as the cytomegalovirus (CMV), influenza virus, and the herpes virus.

Interleukin-6 (IL-6) is a cytokine effecting the immune system and hematopoiesis. It is produced by several mammalian cell types in response to agents such as IL-1, and is correlated with disease states such as angiofollicular lymphoid hyperplasia.

Interleukin-8 (IL-8) is a chemotactic factor first identified and characterized in 1987. Many different names have been applied to IL-8, such as neutrophil attractant/activation protein-1 (NAP-1), monocyte derived neutrophil chemotactic factor (MDNCF), neutrophil activating factor (NAF), and T-cell lymphocyte chemotactic factor. Like IL-1, IL-8 is produced by several cell types, including mononuclear cells, fibroblasts, endothelial cells and ketainocytes. Its production is induced by IL-1, TNF and by lipopolysaccharide (LPS). IL-8 stimulates a number of cellular functions in vitro. It is a chemoattractant for neutrophils, T-lymphocytes and basophils. It induces histamine release from basophils. It causes lysozomal enzyme release and respiratory burst from neutrophils, and it has been shown to increase the surface expression of Mac-1 (CD11b/CD 18) on neutrophils without de novo protein synthesis.

There remains a need for more efficient synthesis of compounds which are effective for treating cancer in which RAF is implicated, as well as compounds which inhibit, suppress or antagonize the production or activity of cytokines such as IL-1, IL-6, IL-8 and TNF. Thus, there is a need for efficient synthesis of intermediates such as acylated keto amides needed to synthesize cytokine inhibiting substituted imidazole compounds.

Inomata et al., Chem. Lett., 1437(1993) describes adding malonate nucleophiles to cyclic sulfonyl amides to generate substituted pyrrolidine. N.J. Liverton, Tetrahedron Lett., 39:8939-8942(1998) describes the preparation of tetrasubstituted imidazoles from N-)2-oxo)-amides. Nevertheless, there remains a need for an efficient method to synthesize intermediates useful to make substituted imidazole compounds.

### SUMMARY OF THE INVENTION

The present invention prepares acylated ketoamides by the addition of acylanion equivalents, either anions of cyano hydrins or through thiazole ylides. The acylated ketoamides are prepared from sulfonyl amides and aldehydes or their related derivatives by a step of

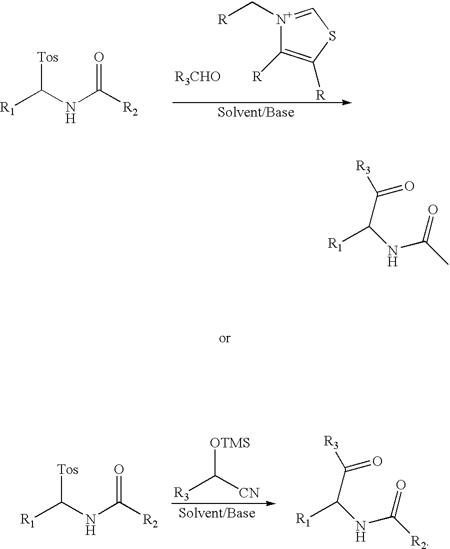

### DETAILED DESCRIPTION OF THE INVENTION

The present invention prepares acylated ketoamides by the addition of acylanion equivalents, either anions of cyano hydrins or through thiazole ylides. The present invention forms acylated ketoamides from sulfonyl amides and aldehydes or their related derivatives.

Generally, the process of the present invention is described by the following chemical reaction scheme:

Step 1

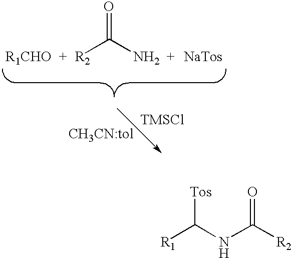

R₁ is hydrogen, C₁₋₆alkyl, C₃₋₇cycloalkyl, or aryl;

R₂ is hydrogen, C₁₋₆alkyl, C₃₋₇cycloalkyl, aryl, heterocyclalkyl, —O—C₁₋₆alkyl, —O-aryl, —NH₂, —NHC₁₋₆alkyl, or —NH-aryl.

Step 2a

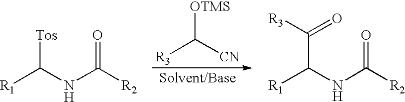

R₃ is C₁₋₆alkyl, C₃₋₇cycloalkyl, aryl, or heteroaryl.

Alternate Step 2b

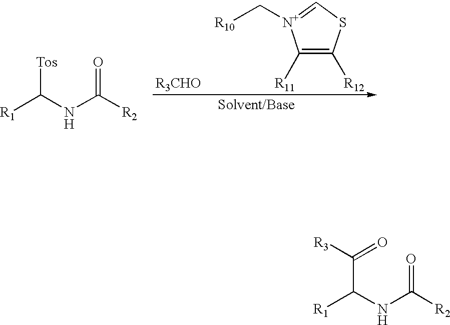

R₁₀, R₁₁, and R₁₂ each independently is hydrogen, C₁₋₆alkyl, C₃₋₇cycloalkyl, or aryl, optionally substituted with OH. An example of the catalyst

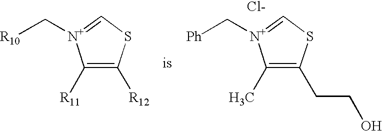

available from Aldrich, Milwaukee, Wis. Other examples of the catalyst are:

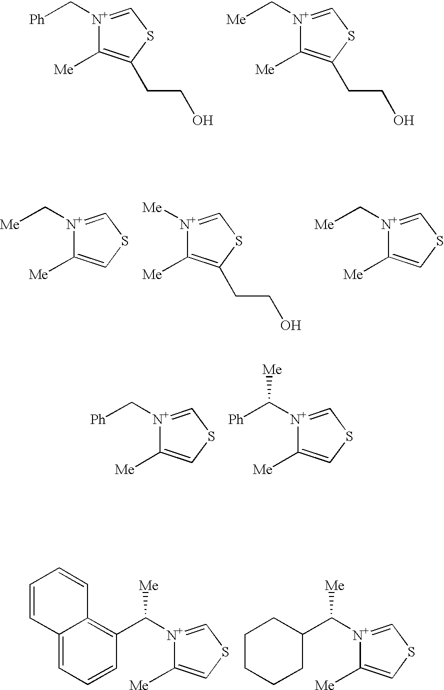

Step 3

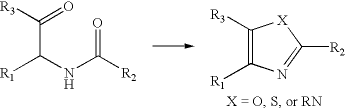

Three Examples are shown below:

### EXAMPLE 1

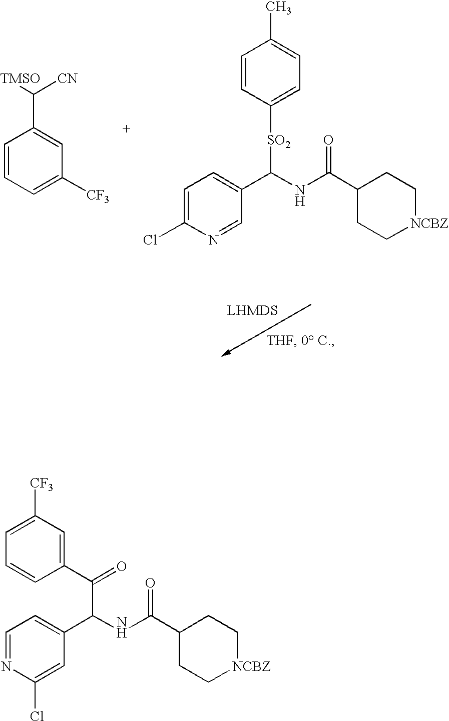

### EXAMPLE 2

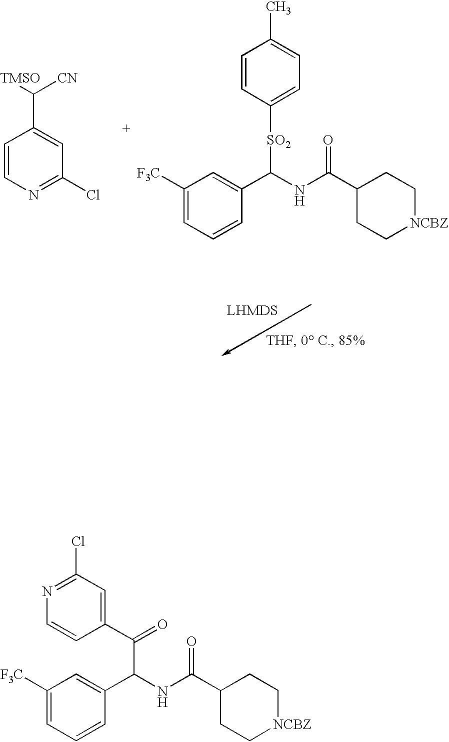

### EXAMPLE 3

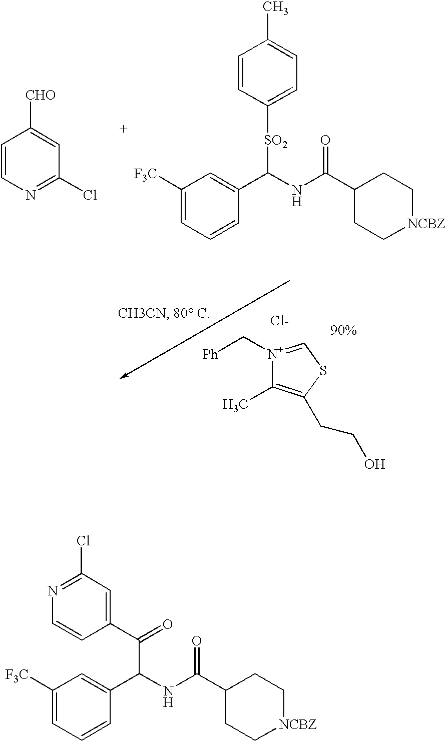

Cyano-Hydrin Synthesis

The cyano-hydrins were prepared as outlined by Kobayashi (Tetrahedron Lett., 34:1901(1993)). To a solution of an aldehyde in methylene chloride was added 0.1equiv of triethylamine and 1.1equiv of trimethylsilyl cyanide. The resulting solution was stirred for 12 h and then concentrated to an oil. The oil was analyzed by NMR and LC and used without further purification.

Cyano-Hydrin Condensation

To a solution of the cyano-hydrin and sulfonyl amide in THF at 0° C. was added 2.2equiv of LHMDS. The resulting solution was allowed to gradually warm to RT and stirred at that temperature to completion. The resulting mixture was diluted with water and ethyl acetate and the layers were then separated. The organic layer was dried over NaSO₄ and concentrated in vacuo to provide an oil which was purified by column chromatography.

General Procedure for the Thiazolium Catalyzed Synthesis of N-Acyl β-Amino Ketones

A flask was charged with the tosyl-amide (1.0eq) and the thiazolium catalyst (0.1eq) and purged with nitrogen for 15 min. To the flask was added an organic solvent (20 mL) followed by the aldehyde (1.1eq) and the resulting mixture stirred and heated to 35° C. Triethylamine (15eq) was added in one portion via syringe and the corresponding reaction was monitored by HPLC analysis for consumption of the tosyl-amide. After the reaction was complete, it was cooled to 25° C. and water was added (20 mL). The resulting layers were separated and the aqueous layer was extracted with CH₂Cl₂ (10 mL). The combined organic layers were washed with brine (10 mL), dried over Na₂SO₄, and concentrated in vacuo. The products were purified by either flash chromatography or crystallization from the crude reaction mixture.

### Example

(2-Oxo-1,2-diphenyl-ethyl)-carbamic acid tert-butyl ester:

A flask was charged with tert-Butyl phenyl(tolylsulfonyl)methylcarbamate (1.0eq) and 3,4-dimethyl-5-(2-hydroxyethyl)thiazolium iodide (0.1eq) and purged with nitrogen for 15 min. To the flask was added CH₂Cl₂ (20 mL) followed by benzaldehyde (1.1eq) and the resulting mixture stirred and heated to 35° C. Triethylamine (15eq) was added in one portion via syringe and the corresponding reaction was monitored by HPLC. After 24 hours it was cooled to 25° C. and water was added (20 mL). The resulting layers were separated and the aqueous layer was extracted with CH₂Cl₂ (10 mL). The combined organic layers were washed with brine (10 mL), dried over Na₂SO₄, and concentrated in vacuo. The product was isolated from the crude reaction mixture by crystallization from ethyl acetate as small white crystals.

mp 113-114° C.

¹H NMR (400 MHz, CDCl₃) δ8.01−7.90 (d, 2H, J=7.6 Hz), 7.53−7.46 (t, 1H, J=7.6 Hz), 7.43−7.34 (t, 4H, J=7.6 Hz), 7.32−7.20 (m, 3H), 6.35−6.25 (d, 1H, J=7.2 Hz), 6.12−6.00 (d, 1H, J=7.2 Hz), 1.50−1.38 (br, 9H).

¹³C NMR (100 MHz, CDCl₃) δ196.1, 155.2, 137.4, 134.5, 133.5, 129.0, 128.5, 128.2, 128.0, 79.8, 59.7, 28.3.

Tosyl Amide Synthesis

A flask was charged with p-toluenesulfinic acid, sodium salt (1.5eq) followed by the amide (1.5eq). The flask was then charged with acetonitrile (500 mL) and the contents were stirred and placed under a positive pressure of nitrogen. To the resulting slurry was added the aldehyde (1.0eq) in one portion. The resulting reaction mixture was then cooled to 10° C. using an ice bath. Then chlorotrimethylsilane (TMSCl) (2.0eq) was slowly added to the reaction mixture to maintain an internal temperature below 25° C. After complete addition of the TMSCl, the reaction was allowed to warm to room temperature (23° C.). The reaction was then monitored by HPLC until completion. To the heterogeneous mixture was added water (500 mL) and the resulting suspension was stirred for 30 min. The solids were isolated by filtration and the filter cake was washed with water (100 mL). The solid was dried in a vacuum oven at 50° C. at 30torr for 24 hours to give the product as a fine white solid.

### Example

tert-Butyl phenyl(tolylsulfonyl)methylcarbamate:

A round-bottomed flask fitted with an addition funnel and an overhead mechanical stirrer was charged with p-toluenesulfinic acid, sodium salt (1.5eq) followed by tert butylcarbamate (1.5eq). The flask was then charged with acetonitrile (500 mL) and the contents stirred and placed under a positive pressure of nitrogen. To the resulting slurry was added benzaldehyde (1.0eq) in one portion. The resulting mixture was then cooled to 10° C. using an ice bath. To the addition funnel was added chlorotrimethylsilane (TMSCl) (2.0eq) and this was slowly added to the reaction mixture to maintain an internal temperature below 25° C. (total addition time was 15 min). After complete addition of the TMSCl, the reaction was allowed to warm to room temperature (23° C.). The reaction was then monitored by HPLC until completion (24 hours). To the heterogeneous mixture was added water (500 mL) and the resulting suspension was stirred for 30 min. The solids were isolated by filtration and the filter cake was washed with water (100 mL). The solid was dried in a vacuum oven at 50° C. at 30torr for 24 hours to give the product as a fluffy white solid.

¹H NMR (400 MHz, CDCl₃) δ7.82−7.78 (d, 2H, J=8.4 Hz), 7.50−7.40 (m, 5H), 7.32-7.38 (d, 2H, J=8.3 Hz), 6.01−5.90 (d, 1H, J=10.4 Hz), 5.86−5.79 (d, 1H, J=10.3 Hz), 2.45−2.40 (s, 3H), 1.40−1.20 (br, 9H);

¹³C NMR (100 MHz, CDCl₃) δ154.0, 144.9, 133.8, 130.0, 129.7, 129.6, 129.4, 128.8, 128.6, 81.0, 73.8, 27.9, 21.5.

tert-Butyl N-(α-tosylbenzyl)carbamate:

¹H NMR (400 MHz, CDCl₃) δ7.82−7.78 (d, 2H, J=8.4 Hz), 7.50−7.40 (m, 5H), 7.32-7.38 (d, 2H, J=8.3 Hz), 6.01−5.90 (d, 1H, J=10.4 Hz), 5.86−5.79 (d, 1H, J=10.3 Hz), 2.45−2.40 (s, 3H), 1.40−1.20 (br, 9H);

¹³C NMR (100 MHz, CDCl₃) δ154.0, 144.9, 133.8, 130.0, 129.7, 129.6, 129.4, 128.8, 128.6, 81.0, 73.8, 27.9, 21.5.

N-(2-Oxo-1,2-diphenyl-ethyl)-carbamic acid tert-butyl ester:

The product was isolated from the crude reaction mixture by crystallization from ethyl acetate as small white needles;

mp 113-114° C.;

¹H NMR (400 MHz, CDCl₃) δ8.01−7.90 (d, 2H, J=7.6 Hz), 7.53−7.46 (t, 1H, J=7.6 Hz), 7.43−7.34 (t, 4H, J=7.6 Hz), 7.32−7.20 (m, 3H), 6.35−6.25 (d, 1H, J=7.2 Hz), 6.12−6.00 (d, 1H, J=7.2 Hz), 1.50−1.38 (br, 9H);

¹³C NMR (100 MHz, CDCl₃) δ196.1, 155.2, 137.4, 134.5, 133.5, 129.0, 128.5, 128.2, 128.0, 79.8, 59.7, 28.3;

Anal. Calcd for C₁₉H₂₁NO₃: C, 73.29; H, 6.80; N, 4.50. Found: C, 72.91; H, 6.76; N, 4.42.

N-(2-Oxo-1-phenyl-2-(2-bromophenyl)-ethyl)-carbamic acid tert-butyl ester:

The product was isolated from the crude reaction mixture by crystallization from (2:1) isopropyl acetate/hexanes as opaque crystals: mp 102-103° C.;

¹H NMR δ7.55−7.50 (d, 1H, J=8.83), 7.31−7.15 (m, 8H), 6.14−6.08 (d, 1H, J=7.23), 6.08−5.98 (d, 1H, J=7.23), 1.50−1.32 (s, 9H);

¹³C NMR δ198.7, 154.8, 138.8, 133.8, 132.0, 129.3, 129.0, 128.5, 128.1, 127.1, 119.7, 80.1, 63.2, 28.4;

Anal. Calcd for C₁₉H₂₀BrNO₃: C, 58.47; H, 5.17; N, 3.59.

Found: C, 58.49; H, 5.16; N, 3.46.

N-(2-Oxo-1-phenyl-2-(3-methoxyphenyl)-ethyl)-carbamic acid tert-butyl ester:

The product was isolated from the crude reaction mixture by crystallization from (2:1) isopropylacetate/hexanes as clear prisms:

mp 90-91° C.;

¹H NMR δ7.60−7.51 (d, 1H, J=7.61), 7.48−7.42 (s, 1H), 7.38−7.18 (m, 6H), 7.08−7.00 (dd, 1H, J=8.41, J=2.40), 6.30−6.20 (d, 1H, J=7.21), 6.06−5.95 (d, 1H, J=7.21), 3.83−3.76 (s, 3H), 1.52−1.31 (s, 9H);

¹³C NMR δ28.3, 55.3, 59.8, 79.8, 113.0, 120.2, 121.6, 128.0, 128.2, 129.0, 129.5, 135.8, 137.5, 154.9, 159.7, 195.8;

Anal. Calcd for C₂₀H₂₃NO₄: C, 70.36; H, 6.79; N, 4.10. Found: C, 70.31; H, 6.89; N, 3.99.

N-(2-oxo-1-phenyl-2-pyridin-4-yl-ethyl)-carbamic acid tert-butyl ester:

The product was isolated from the crude reaction mixture by flash chromatography using (9:1) ethyl acetate/hexanes as eluent to afford the product as a white crystalline solid;

mp 135-136° C.;

¹H NMR δ8.72−8.66 (d, 2H, J=6.01), 7.70−7.62 (d, 2H, J=6.01), 7.34−7.20 (m, 5H), 6.22−6.16 (d, 1H, J=7.21), 6.01−5.90 (d, 1H, J=7.21), 1.50−1.32 (s, 9H);

¹³C NMR δ195.9, 154.9, 150.8, 140.7, 135.8, 129.3, 128.7, 128.1, 121.5, 80.1, 60.4, 28.2;

Anal. Calcd for C₁₈H₂₀N₂O₃: C, 69.21; H, 6.45; N, 8.97. Found: C, 68.97; H, 6.43; N, 8.82.

N-(2-oxo-1-phenyl-propyl)-carbamic acid tert-butyl ester:

The product was isolated from the crude reaction mixture by crystallization from (1:1) methylene chloride/hexanes;

mp 82-83° C.;

¹H NMR δ7.28-7.40 (m, 5H), 5.83-5.90 (s, 1H), 5.22-5.29 (d, 1H, J=6.02 Hz), 2.02-2.08 (s, 3H), 1.22-290 1.45 (s, 9H);

¹³C NMR δ27.0, 28.3, 64.8, 79.9, 127.9, 128.5, 129.2, 137.0, 154.9, 203.6;

Anal. Calcd for C₁₄H₁₉NO₃: C, 67.45; H, 7.68; N, 5.62. Found: C, 67.50; H, 7.79; N, 5.53.

N-(3-Benzyloxy-2-oxo-1-phenyl-propyl)-carbamic acid tert-butyl ester:

The product was isolated from the crude reaction mixture by crystallization from (1:1) methylene chloride/hexanes;

mp 60-61° C.;

¹H NMR δ7.20-7.40 (m, 10H), 6.78-6.84 (s, 1H), 5.55-6.0 (d, 1H, J=7.23 Hz), 4.35-4.52 (dd_{AB}, 2H, J_{AB}=11.64 Hz), 4.02-4.10 (s, 1H), 1.22-1.45 (s, 9H);

¹³C NMR 28.4, 61.2, 72.7, 73.5, 79.9, 128.0 (2C), 128.1, 128.5, 128.6, 129.2, 136.2, 136.9, 154.7, 204.2;

Anal. Calcd for C₂₁H₂₅NO₄: C, 70.96; H, 7.09; N, 3.94. Found: C, 70.73; H, 7.16; N, 3.87.

4-Methoxy-N-(2-oxo-1-phenyl-2-pyridin-4-yl-ethyl)-benzamide:

The product was isolated from the crude reaction mixture by filtering through silica gel with several washing of ethyl acetate to yield the product:

m.p. 103-105° C.;

¹H NMR δ8.76−8.79 (d, 2H, J=4.41 Hz), 7.81-7.83 (dd, 2H, J=6.81 Hz, J=2.00 Hz,), 7.74−7.76 (d, 2H, J=4.41 Hz), 7.27-310 47 (m, 6H), 6.91-6.95 (dd, 2H, J=6.81 Hz, J=2.00 Hz), 6.67-6.69 (d, 1H, J=6.81 Hz), 3.87 (s, 3H);

¹³C NMR δ55.3, 59.5, 113.7, 121.6, 125.7, 128.4, 128.8, 129.0, 129.4, 135.7, 140.6, 150.9, 162.4, 166.0, 195.8;

Anal. Calcd for C₂₁H₁₈N₂O₃: C, 72.82; H, 5.24; N, 8.09. Found: C, 72.21; H, 5.23; N, 7.87.

4-Fluoro-N-(2-oxo-1-phenyl-2-pyridin-4-yl-ethyl)-benzamide:

The product was isolated from the crude reaction mixture by filtering through silica gel with several washing of ethyl acetate to yield the product:

m.p. 131-132° C.;

¹H NMR δ 8.77 (d, 2H, J=4.41 Hz), 7.82-7.90 (m, 2H), ), 7.74-7.76 (d, 2H, J=4.41 Hz), 7.41-7.49 (m, 3H), 7.28-7.39 (m, 3H), 7.08-7.16 (t, 2H, J=8.41 Hz), 6.63-6.67 (d, 1H, J=6.81 Hz);

¹³C NMR δ59.6, 115.5, 115.7, 121.6, 128.4, 129.0, 129.5, 129.6, 135.5, 140.4, 150.9, 163.6, 165.4, 166.1, 195.6;

Anal. Calcd for C₂₀H₁₅FN₂O₂: C, 71.85; H, 4.52; N, 8.38. Found: C, 71.37; H, 4.58; N, 8.15.

N-(2-oxo-1-phenyl-2-pyridin-4-yl-ethyl)-carbamic acid benzyl ester:

The product was isolated from the crude reaction mixture by chromatography over silica gel (60% EtOAc/40% Hexane) to yield the product:

m.p. 95-96° C.;

¹H NMR δ8.70-8.77 (d, 2H, J=6.01 Hz), 7.65-7.71 (d, 2H, J=6.01 Hz), 7.28-7.42 (m, 10H), 6.20-6.26 (d, 1H, J=6.81 Hz), 6.15-6.19 (d, 1H, J=6.81 Hz), 5.03-5.17 (dd_{AB}, 2H, J_{AB}=12.01 Hz);

¹³C NMR δ60.8, 67.1, 121.6, 128.1, 128.2, 128.5, 128.9, 129.4, 135.7, 136.0, 140.4, 150.8, 155.4, 195.3;

Anal. Calcd for C₂₁H₁₈N₂O₃: C, 72.82; H, 5.24; N, 8.09. Found: C, 72.75; H, 5.18; N, 7.91.

N-(2-oxo-1-phenyl-2-pyridin-4-yl-ethyl)-acetamide:

The product was isolated from the crude reaction mixture by chromatography over silica gel (98% EtOAc/2% MeOH) to yield 94% of product:

m.p. 142-144° C.;

¹H NMR δ8.72-8.77 (d, 2H, J=4.41 Hz), 7.65-7.72 (d, 2H, J=4.41 Hz), 7.27-7.38 (m, 5H), 6.75-6.83 (d, 1H, J=7.21 Hz), 6.43-6.50 (d, 1H, J=7.21 Hz), 2.02-351 2.10 (s, 3H);

¹³C NMR δ22.9, 59.1, 121.6, 128.2, 128.8, 129.4, 135.6, 140.5, 150.8, 169.3, 195.6;

Anal. Calcd for C₁₅H₁₄N₂O₂: C, 70.85; H, 5.55; N, 11.02. Found: C, 70.63; H, 5.50; N, 10.76.

N-(2-oxo-1-phenyl-2-pyridin-4-yl-ethyl)-formamide:

The product was isolated from the crude reaction mixture by chromatography over silica gel (100% EtOAc) to yield the product: m.p. 134-135° C.;

¹H NMR δ8.72-8.78 (d, 2H, J=4.41 Hz), 8.27 (s, 1H), 7.69-7.71 (d, 2H, J=4.41 Hz), 7.27-7.39 (m, 5H), 7.05-7.13 (d, 1H, J=7.21 Hz), 6.52-6.57 (d, 1H, J=7.21 Hz);

¹³C NMR δ57.8, 121.6, 128.2, 129.0, 129.5, 135.3, 140.2, 150.9, 160.1, 194.9;

Anal. Calcd for C₁₄H₁₂N₂O₂: C, 69.99; H, 5.03; N, 11.66.

Found: C, 69.63; H, 5.02; N, 11.41.

N-(2-oxo-1-phenyl-2-pyridin-4-yl-ethyl)-benzamide:

The product was isolated from the crude reaction mixture by chromatography over silica gel (70% EtOAc/30% Hexane) to yield 88% of product:

m.p. 144-145° C.;

¹H NMR δ8.73-8.80 (d, 2H, J=4.41 Hz), 7.81-7.86 (m, 2H), 7.74-7.76 (d, 2H, J=4.41 Hz), 7.41-7.56 (m, 6H), 7.28-7.39 (m, 3H), 6.65-6.70 (d, 1H, J=6.81 Hz);

¹³C NMR δ59.6, 121.6, 127.1, 128.4, 128.5, 128.9, 129.5, 131.8, 133.4, 135.6, 140.5, 150.9, 166.5, 195.6;

Anal. Calcd for C₂₀H₁₆N₂O₂: C, 75.93; H, 5.10; N, 8.86. Found: C, 75.67; H, 5.05; N, 8.70.

N-(2-oxo-l-phenyl-2-pyridin-4-yl-ethyl)-cyclohexanecarboxamide:

The product was isolated from the crude reaction mixture by chromatography over silica gel (70% EtOAc/30% Hexane) to yield the product:

m.p. 162-163° C.;

¹H NMR δ8.70-8.74 (d, 2H, J=4.41 Hz), 7.69-7.71 (d, 2H, J=4.41 Hz), 7.27-7.48 (m, 5H), 6.65-6.72 (d, 2H, J=7.21 Hz), 6.43-6.48 (d, 2H, J=7.21 Hz), 2.2 (tt, 1H, J=11.61 Hz, J=3.20 Hz), 1.61-1.95 (m, 5H), 1.35-1.50 (m, 2H), 1.15-1.34 (m, 3H);

¹³C NMR δ25.5 (2 C), 25.6, 29.3, 29.4, 44.9, 58.9, 121.6, 128.2, 128.7, 129.4, 135.7, 140.7, 150.8, 175.3, 195.9;

Anal. Calcd for C₂₀H₂₂N₂O₂: C, 74.51; H, 6.88; N, 8.69. Found: C, 74.41; H, 6.94; N, 8.65.

N-(2-oxo-1-phenyl-2-pyridin-2-yl-ethyl)-cyclohexanecarboxamide:

The product was isolated from the crude reaction mixture by chromatography over silica gel (60% CH₂Cl₂/30% EtOAc/10% Hexane) to yield the product:

m.p. 148-149° C.;

¹H NMR δ8.63-8.69 (d, 1H, J=4.81 Hz), 7.98-8.03 (d, 1H, J=7.61 Hz), 7.72-7.80 (dt, 1H, J=7.61 Hz, J=1.60 Hz), 7.42-7.49 (d, 2H, J=7.21 Hz 7.37-7.42 (dd, 1H, J=7.61 Hz, J=4.81 Hz), 7.21-7.29 (t, 2H, J=7.21 Hz), 7.19-7.21 (d, 1H, J=7.21 Hz), 7.10-7.15 (d, 1H, J=7.21 Hz), 6.80-6.90 (d, 1H, J=7.21 Hz), 2.14-2.24 (tt, 1H, J=11.61 Hz, J=3.20 Hz), 1.61-1.95 (m, 5H), 1.35-1.52 (m, 2H), 1.15-1.34 (m, 3H);

¹³C NMR δ25.6 (2 C), 25.7, 29.4, 29.5, 45.1, 57.3, 123.1, 127.4, 127.8, 128.3, 128.5, 128.6, 136.7, 136.9, 148.9, 151.2, 175.0, 196.9;

Anal. Calcd for C₂₀H₂₂N₂O₂: C, 74.51; H, 6.88; N, 8.69. Found: C, 74.38; H, 6.96; N, 8.56.

N-(2-oxo-1-phenyl-2-pyridin-3-yl-ethyl)-carbamic acid tert-butyl ester:

The product was isolated from the crude reaction mixture by chromatography over silica gel (60% EtOAc/40% Hexane) to yield the product:

m.p. 113-114° C.;

¹H NMR δ9.13-9.16 (dd, 1H, J=2.41 Hz, J=0.80 Hz), 8.68-8.71 (dd, 1H, J=4.82 Hz, J=1.61 Hz), 8.18-8.21 (td, 1H, J=8.03 Hz, J=1.61 Hz), 7.27-7.40 (m, 6H), 6.18-6.22 (d, 1H, J=7.23 Hz), 5.90-5.95 (d, 1H, J=7.23 Hz), 1.30-1.48 (s, 9H);

¹³C NMR δ28.2, 60.3, 80.1, 123.5, 128.1, 128.6, 129.3, 130.0, 136.1, 136.3, 150.2, 153.2, 154.9, 195.1;

Anal. Calcd for C₁₈H₂₀N₂O₃: C, 69.21; H, 6.45; N, 8.97. Found: C, 68.61; H, 6.48; N, 8.78.

N-(2-Furan-2-yl-2-oxo-1-phenyl-ethyl)-carbamic acid tert-butyl ester:

The product was isolated from the crude reaction mixture by crystallizition from isopropyl acetate to yield the product: m.p. 110-111° C.;

¹H NMR δ7.55-7.56 (dd, 1H, J=1.61 Hz, J=0.80 Hz), 7.38-7.42 (m, 2H), 7.26-7.36 (m, 3H), 7.24-7.26 (dd, 1H, J=3.61 Hz, J=0.80 Hz), 6.47-6.49(dd, 1H, J=3.61 Hz, J=1.61 Hz), 6.02-6.08 (d, 1H, J=7.23 Hz), 5.87-5.95 (d, 1H, J=7.23 Hz), 1.30-1.50 (s, 9H);

¹³C NMR δ25.2, 59.8, 79.9, 112.4, 119.2, 127.9, 128.2, 128.9, 137.0, 147.0, 150.6, 154.8, 184.7;

Anal. Calcd for C₁₇H₁₉NO₄: C, 67.76; H, 6.36; N, 4.65. Found: C, 67.47; H, 6.46; N, 4.56.

N-[2-(4-cyano-phenyl)-2-oxo-1-phenyl-ethyl]-carbamic acid tert-butyl ester:

The product was isolated from the crude reaction mixture by chromatography over silica gel (70% EtOAc/30% Hexane) to yield the product:

m.p. 115-117° C.;

¹H NMR δ7.98-8.02 (d, 2H, J=8.41 Hz), 7.68-7.71 (d, 2H, J=8.41 Hz), 7.27-7.35 (m, 5H), 6.20-6.24 (d, 1H, J=7.21 Hz), 5.82-5.90 (d, 1H, J=7.21 Hz), 1.35-1.50 (s, 9H);

¹³C NMR δ28.2, 60.3, 80.2, 116.6, 117.6, 128.1, 128.7, 129.2, 129.4, 132.4, 136.1, 137.7, 154.9, 195.1;

Anal. Calcd for C₂₀H₂₀N₂₃: C, 71.41; H, 5.99; N, 8.33. Found: C, 71.34; H, 5.96; N, 8.10.

N-(2-oxo-1,4-diphenyl-trans-but-3-enyl)-cyclohexanecarboxamide:

The product was isolated from the crude reaction mixture by crystallizition from hot ethanol to yield the product:

m.p. 183-185° C.;

¹H NMR δ7.69-7.73 (d, 1H, J=16.02 Hz), 7.46-7.49 (dd, 2H, J=8.01 Hz, J=2.00 Hz), 7.29-7.40 (m, 8H), 6.95-7.00 (d, 1H, J=6.41 Hz), 6.68-6.74(d, 1H, J =16.02 Hz), 5.82-5.88 (d, 1H, J=6.41 Hz), 2.14-2.24 (m, 1H), 1.65-1.93 (m, 5H), 1.35-1.51 (m, 2H), 1.15-1.33 (m, 3H);

¹³C NMR δ25.6 (3 C), 29.4 (2 C), 45.1, 61.5, 122.4, 128.1, 128.3, 128.5, 128.8, 129.1, 130.9, 133.9, 136.8, 144.6, 175.2, 194.5;

Anal. Calcd for C₂₃H₂₅NO₂: C, 79.51; H, 7.25; N, 4.03. Found: C, 78.79; H, 7.26; N, 3.92.

What is claimed is:

1. A method of forming a reaction product mixture substantially containing 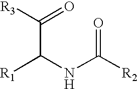
wherein
R₁ is hydrogen, C₁₋₆alkyl, C₃₋₇cycloalkyl, or aryl;
R₂ is hydrogen, C₁₋₆alkyl, C₃₋₇cycloalkyl, aryl, heterocyclalkyl, —O—C₁₋₆alkyl, —O-aryl, —NH₂, —NHC₁₋₆alkyl, or —NH-aryl;
R₃ is C₁₋₆alkyl, C₃₋₇cycloalkyl, aryl, or heteroaryl;
said method comprising:
reacting 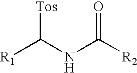
in an effective amount of R₃CHO or an effective amount of 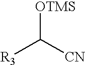
in an effective amount of a basic solvent.

2. A method of forming a reaction product mixture substantially containing 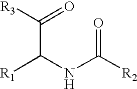
wherein
R₁ is hydrogen, C₁₋₆alkyl, C₃₋₇cycloalkyl, or aryl;
R₂ is hydrogen, C₁₋₆alkyl, C₃₋₇cycloalkyl, aryl, heterocyclalkyl, —O—C₁₋₆alkyl, —O-aryl, —NH₂, —NHC₁₋₆alkyl, or —NH-aryl;
R₃ is C₁₋₆alkyl, C₃₋₇cycloalkyl, aryl, or heteroaryl;
said method comprising:
reacting 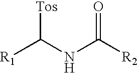
in effective amount of R₃CHO and an effective amount of a catalyst 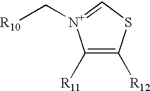
wherein R₁₀, R₁₁, and R₁₂ each independently is hydrogen, C₁₋₆alkyl, C₃₋₇cycloalkyl, or aryl, optionally substituted with OH.

3. The method of claim 2, wherein said catalyst is

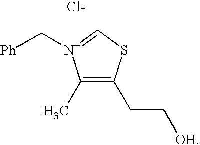

4. The method of claim 2, wherein said catalyst is [chemical structure]
